(19) United States
(12) Patent Application Publication
(10) Pub. No.: US 2021/0228448 A1
(43) Pub. Date: Jul. 29, 2021

(54) MEDICATION REMINDER METHOD, NON-TRANSITORY READABLE STORAGE MEDIUM STORING SAID METHOD, AND MEDICATION REMINDER DEVICE

(71) Applicant: BOE TECHNOLOGY GROUP CO., LTD., Beijing (CN)

(72) Inventor: Litong Han, Beijing (CN)

(73) Assignee: BOE TECHNOLOGY GROUP CO., LTD., Beijing (CN)

(21) Appl. No.: 16/301,567

(22) PCT Filed: Mar. 14, 2018
(86) PCT No.: PCT/CN2018/078905

§ 371 Date: Nov. 14, 2018

(30) Foreign Application Priority Data
Oct. 30, 2017 (CN) 201711032427.X

(51) Int. Cl. A61J 7/04; A61B 5/00; A61B 5/145; G16H 50/30; G16H 10/60; G16H 20/10; G16H 70/40; G16H 50/70; G16H 40/67

(52) U.S. Cl. CPC A61J 7/0481; A61J 7/0418; A61B 5/681; A61B 5/145; A61B 5/4833; G16H 40/67; G16H 10/60; G16H 20/10; G16H 70/40; G16H 50/70; G16H 50/30

(57) ABSTRACT

A method, system, and non-transitory computer-readable storage medium storing a method for reminding a patient to comply with medication schedule. The method includes obtaining a measurement of at least one patient parameter; determining whether the measurement is outside of a range; when the measurement is outside of the range, determining a total time lapse since the previous administration of a dose of the medication; determining a concentration of the medication in blood of the patient based at least on the total time lapse; and when the concentration of the medication in the blood of the patient is below a threshold, generating a reminder to the patient to administer the next dose of the medication.

### CROSS-REFERENCE TO RELATED APPLICATIONS

This application claims the benefit of the filing date of Chinese Patent Application No. 201711032427.X filed on Oct. 30, 2017, the entire disclosure of which is hereby incorporated by reference.

### TECHNICAL FIELD

The present disclosure generally relates to the field of medical systems, and in particular, to a method, system, and non-transitory computer-readable storage medium storing a method for timely reminding a patient to comply with a medication dosage schedule.

### BACKGROUND

A patient's compliance with a medication schedule is generally paramount in the effectiveness of a therapeutic regimen to combat an illness. This may be especially the case when the illness is of a chronic nature, such as diabetes or hypertension, the management of which relies on the continued administration of medications. Non-compliance with the therapeutic regimen can have considerable disruptive consequences. Unfortunately, patients often fail to adhere to prescribed treatment programs. Non-adherence is particularly prevalent among older patients, which may be due to those patients' increased forgetfulness.

### BRIEF SUMMARY

One embodiment of the present disclosure is a method for providing a reminder to a patient of when a second dose of a medication should be administered, the patient having been administered a first dose of the medication.

The method may comprise obtaining a measurement of at least one patient parameter; determining whether the measurement of the at least one patient parameter is outside of a range; when the measurement of the at least one patient parameter is outside of the range, determining a total time lapse since the administration of the first dose of the medication; determining a concentration of the medication in blood of the patient based at least on the total time lapse; and when the concentration of the medication in the blood of the patient is below a threshold, generating a reminder to the patient to administer the second dose of the medication. In at least some embodiments, the at least one patient parameter characterizes a medical condition to be treated by the medication.

In at least some embodiments, the method may further comprise retrieving a pharmacokinetics profile of the medication, wherein the concentration of the medication in the blood of the patient is determined based on the total time lapse and the pharmacokinetics profile of the medication.

In at least some embodiments, the method may further comprise obtaining a profile of the patient. The profile may comprise information relating to at least one of an age and a gender of the patient. The retrieved pharmacokinetics profile of the medication may be specific to at least one of the age and the gender of the patient.

In at least some embodiments, the method may further comprise receiving, at a time of the administration of the first dose of the medication, an indication indicating the time of the administration. In at least some embodiments, the method further comprises determining the total time lapse based on the time of the administration of the first dose of the medication.

In at least some embodiments, the method may further comprise obtaining an image of a packaging of the medication. The generated reminder may comprise the image of the packaging of the medication.

Another embodiment of the present disclosure is a device for providing a reminder to a patient of when a second dose of a medication should be administered, the patient having been administered a first dose of the medication.

The device may comprise a parameter acquisition unit configured to obtain a measurement of at least one patient parameter, and determine whether the measurement of the at least one patient parameter is outside of a range; a time counter configured to, when the measurement of the at least one patient parameter is outside of the range, determine a total time lapse since the administration of the first dose of the medication; a concentration determination unit configured to determine a concentration of the medication in blood of the patient based at least on the calculated total time lapse; and a reminder generation unit configured to, when the concentration of the medication in the blood of the patient is below a threshold, generate a reminder to the patient to administer the second dose of the medication. The at least one patient parameter may characterize a medical condition to be treated by the medication.

In at least some embodiments, the concentration determination unit may be further configured to acquire a pharmacokinetics profile of the medication, so as to determine the concentration of the medication in the blood of the patient based on the total time lapse and the pharmacokinetics profile of the medication.

In at least some embodiments, the reminder generation unit may be further configured to obtain an image of a packaging of the medication, and configured to generate a reminder that comprises the image of the packaging of the medication.

In at least some embodiments, the device may further comprise a receiving unit configured to receive, at a time of the administration of the first dose of the medication, an indication indicating the time of the administration. The time counter may be configured to determine the total time lapse based on the time of the administration of the first dose of the medication.

Another embodiment of the present disclosure is a system for providing a reminder to a patient of when a second dose of a medication should be administered, the patient having been administered a first dose of the medication. The system may comprise a medication reminder device as described herein, and a server coupled to the medication reminder device.

In at least some embodiments, the concentration determination unit may be further configured to acquire medication information of the patient. The medication information including may identify information of the medication. The concentration determination unit may be further configured to transmit to the server a request for a pharmacokinetics profile of the medication. The request may include the identifying information of the medication. In at least some embodiments, the server may be configured to retrieve the pharmacokinetics profile of the medication based on the identifying information of the medication, and to transmit the pharmacokinetics profile of the medication to the concentration determination unit for determining the concentration of the medication in the blood of the patient.

In at least some embodiments, the concentration determination unit may be further configured to acquire medication information of the patient. The medication information may include identifying information of the medication. The concentration determination unit may be further configured to transmit to the server a request for a determination of the concentration of the medication in the blood of the patient. The request may include the identifying information of the medication and the total time lapse. In at least some embodiments, the server may be configured to: retrieve a pharmacokinetics profile of the medication based on the identifying information of the medication, determine the concentration of the drug in the blood of the patient based on the total time lapse and the pharmacokinetics profile of the medication, and transmit the drug concentration determination to the medication reminder device.

In at least some embodiments, the concentration determination unit may be further configured to: acquire medication information of the patient, the medication information including identifying information of the medication, based on the identifying information of the medication, retrieve a pharmacokinetics profile of the medication, and determine the concentration of the drug in the blood of the patient based on the total time lapse and the pharmacokinetics profile of the medication.

In at least some embodiments, the medication reminder device may be provided in a portable device which is coupled to a wearable device. The wearable device may comprise a sensor configured to measure the at least one patient parameter.

In at least some embodiments, the medication reminder device may be provided in a portable device which is coupled to a protective housing. The protective housing may be configured to house the portable device. The protective housing may comprise a sensor configured to measure the at least one patient parameter.

Another embodiment of the present disclosure is a non-transitory computer-readable storage medium storing a program that, when executed by a computer, performs the method as described herein.

### BRIEF DESCRIPTION OF THE DRAWINGS

The subject matter which is regarded as the invention is particularly pointed out and distinctly claimed in the claims at the conclusion of the specification. The foregoing and other objects, features, and advantages of the present disclosure are apparent from the following detailed description taken in conjunction with the accompanying drawings in which:

FIGS. 1a to 1c show a network structure comprising a medication reminder device according to embodiments of the present disclosure.

FIGS. 2 and 3 are flow diagrams of a method according to embodiments of the present disclosure.

FIGS. 4 and 5 are block diagrams of a medication reminder device according to embodiments of the present disclosure.

FIGS. 6 and 7 are block diagrams of a medication reminder system comprising a medication reminder device, according to embodiments of the present disclosure.

FIG. 8 is a schematic diagram of a terminal device according to an embodiment of the present disclosure.

FIG. 9 is a schematic diagram of a server according to an embodiment of the present disclosure.

### DETAILED DESCRIPTION

Next, the embodiments of the present disclosure will be described clearly and concretely in conjunction with the accompanying drawings, which are described briefly above. The subject matter of the present disclosure is described with specificity to meet statutory requirements. However, the description itself is not intended to limit the scope of this disclosure. Rather, the inventors have contemplated that the claimed subject matter might also be embodied in other ways, to include different steps or elements similar to the ones described in this document, in conjunction with other present or future technologies.

While the present technology has been described in connection with the embodiments of the various figures, it is to be understood that other similar embodiments may be used or modifications and additions may be made to the described embodiments for performing the same function of the present technology without deviating therefrom. Therefore, the present technology should not be limited to any single embodiment, but rather should be construed in breadth and scope in accordance with the appended claims. In addition, all other embodiments obtained by one of ordinary skill in the art based on embodiments described in this document are considered to be within the scope of this disclosure.

Some conventional systems can monitor a patient's medication compliance by periodically measuring one or more vital parameter, and a measurement that falls outside of a certain predetermined range triggers the systems to remind the patient to take the medication. An example of such conventional systems is the periodic measure of the blood pressure of a patient suffering from hypertension, and when the blood pressure exceeds 140 mmHg, the system alerts the patient to take their blood pressure medication.

However, conventional technologies have certain shortcomings. Certain activities can cause a patient's vital parameters to fluctuate temporarily beyond the norms. An example of such fluctuations is the temporary increase in the blood pressure of a patient suffering from hypertension resulting from body movements. Those temporary fluctuations could trigger a conventional system, such as that described above, to generate an incorrect reminder to a patient to take medication. Such false alarms causing the patient to medicate outside of the scheduled times could compromise the effectiveness of the therapeutic program.

To reduce the risk of patients' non-compliance with therapeutic medication regimens, a variety of medication reminder systems have been developed. These systems specifically monitor a patient's vital parameters, and when one or more of the patient's vital parameters are outside of the predetermined range, generate an alert to remind the patient to take the appropriate medication.

However, in practice, the existing systems are prone to false alarms. Several factors can cause a patient's vital parameters to fluctuate, including physical exercise, bodily position, and/or diet. In other words, that the patient's vital parameters are outside of predetermined ranges at a given moment is not necessarily determinative of the level of drug in the patient's body. However, relying on measurements of the patient's vital parameters to determine the appropriateness of reminders, the existing systems are liable to generate false reminders. Not only are such false reminders dangerous for exposing the patient to the risk of overdose, but they are also undesirable for interfering with the patient's treatment, the effectiveness of which often requires that the patient adhere to a strict medication schedule.

The medication reminder method and system of the present disclosure have been designed to solve the technological problems associated with the existing technologies, including the specific problems described above. The medication reminder system described in the present disclosure significantly improves on conventional technologies. In a basic aspect, the medication reminder system of the present disclosure determines whether one or more patient parameter is outside of a desired range, which may be predetermined. This information is used to determine the total time lapse since the previous administration of a dose of the drug. The concentration of the drug in the patient's blood serum is then determined according to the total time lapse. If the drug concentration in the patient's blood serum falls below a threshold, which may also be predetermined, the medication reminder system reminds the patient to administer a dose of the drug. In other words, the medication reminder system of the present disclosure generates a reminder when the one or more patient parameter is outside of the desired range and the drug concentration in the patient's blood serum falls below a particular threshold. A lower-than-threshold drug concentration in the blood serum signifies that the level of the drug in the patient's body is no longer sufficient to maintain the patient parameter within the desired range. An advantage of linking the generation of a reminder to the concentration of the drug in the patient's blood serum is to reduce the risk of false reminders as a result of temporary fluctuations in the patient parameter (for example, during periods of exercise or other body movements).

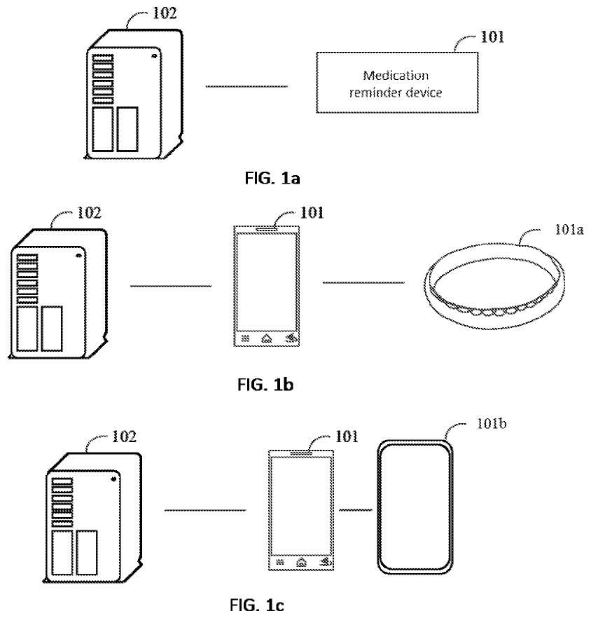

FIG. 1a shows an implementation of an embodiment of the medication reminder method according to the present disclosure. As shown in FIG. 1a, a medication reminder device 101 is coupled to a server 102. The coupling may be wired or wireless. The medication reminder device 101 may be provided in a portable device (for example, a mobile phone, a tablet, and the like) or a wearable device (for example, a smart bracelet, a smart watch, smart glasses, other wearable electronics that can be worn on the body as implants or accessories, and the like). In a preferred embodiment, when the medication reminder device 101 is provided in a wearable device, the wearable device comprises a sensor that is configured to monitor one or more patient parameter. The parameter being monitored may be a vital parameter, for example, body temperature, pulse rate, respiration rate, blood pressure, and blood glucose level. In a preferred embodiment, when the medication reminder device 101 is provided in a portable device, a medication reminder software application is installed on the portable device, and the medication reminder software application is configured to execute the medication reminder method according to the present disclosure.

As shown in FIG. 1b, when the medication reminder device 101 is provided in a portable device, the medication reminder device 101 may be coupled to a wearable device 101a. Alternatively, as shown in FIG. 1c, when the medication reminder device 101 is provided in a portable device, the medication reminder device 101 may be coupled to a protective housing 101b. The protective housing 101b houses the portable device comprising the medication reminder device 101. In some embodiments, the wearable device 101a and the protective housing 101b comprise a sensor that is configured to monitor one or more patient parameter.

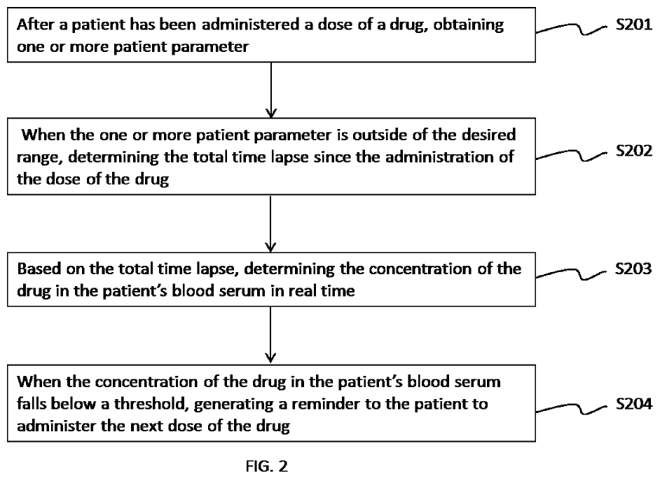

FIG. 2 is a flow diagram of a medication reminder method according to an embodiment of the present disclosure.

In step 201 (S201), after a patient is administered a dose of a drug, the medication reminder device obtains one or more patient parameter. The one or more patient parameter is used to characterize the medical condition being treated by the drug.

In step 202 (S202), when the one or more patient parameter is outside of a desired range, which range may be predetermined, the medication reminder device determines the total time lapse since the administration of the dose of the drug.

In step 203 (S203), based on the total time lapse, the medication reminder device determines the concentration of the drug in the patient's blood serum in real time.

In one embodiment of the present disclosure, and without being limited by any particular theory, a drug's pharmacokinetics profile shows the blood concentration of the drug (c) plotted as a function of time (t), that is, as represented by the functional relationship c=f(t), and generally, the profile is characterized by an early steep increase in the blood concentration of the drug until the concentration reaches a peak, after which the concentration of the drug in the blood slowly decreases to below a therapeutically effective level. This level therefore marks the threshold above which the blood concentration of the drug must be maintained in order for the drug to exert its therapeutic effects. The dose-response curve of the drug can be graphed with the measured effect to the drug (for example, a measured patient parameter) (e) as a function of the blood concentration of the drug (c), that is, as represented by the functional relationship e=f(c). Below a certain blood concentration (c), the drug will have no medically recognizable effects on the patient. Conversely, above a certain blood concentration (c), the drug will exert the therapeutic effects for which it is medically indicated. The dose-response curve of a drug can have a steeper slope than the pharmacokinetics profile of the same drug. A reason may be that, in practice, data for generating the pharmacokinetics profile can be difficult to obtain. A drug is oftentimes administered at a dose level that has been shown during clinical development to demonstrate clinically relevant and statistically significant response.

In some embodiment of the present disclosure, the amount of drug in a patient's blood serum can be determined as shown in the following illustrative and non-limiting example. In this example, for a patient with hyperglycemia, the patient's blood glucose level h is monitored and plotted against time t by a sensor. Time t₀ marks the first administration of the relevant drug, and at time t₁, the patient's blood glucose level h may exceed a critical level. This surge in the patient's blood glucose level h may have one of two causes. In the first scenario, the patient's sudden movement may cause a sudden increase in the blood sugar level. In the second scenario, the dose of the drug during the first administration was too low to be effective. In the first scenario, a medication reminder device according to the present disclosure may be configured to compare the blood glucose level-time curve obtained by the sensor against the pharmacokinetics profile of the drug. If the blood concentration of the drug at time t₁ is still above the threshold clinically determined as necessary to obtain the desired therapeutic effects, then the measured increase in the patient's blood glucose level h may be discarded as the result of inadvertent fluctuation due to the patient's body movements, and no false reminders will be generated by the medication reminder device according to the present disclosure. On the other hand, if it is determined that the increase in the patient's blood glucose level h is the result of insufficient dose during the first administration, for example, the blood concentration of the drug at time t₁ is below the clinically determined threshold, then the medication reminder device according to the present disclosure will generate a reminder.

In step 204 (S204), when the concentration of the drug in the patient's blood serum falls below a threshold, the medication reminder device generates a reminder to the patient to administer the next dose of the drug.

As an illustrative, non-limiting example of an embodiment of the present disclosure, in the case of a fever, a patient may be prescribed CONTAC® and other drugs to treat fever reaching about 38 degrees. Drugs such as CONTAC® are slow-releasing (approximately 5 hours in most cases), so that the medication regimen to bring the temperature down to 37 degrees often involves longer intervals between each dosage. In contrast, more fast-acting drugs such as ibuprofen are released more rapidly, and the drugs can usually reduce the fever within 2 hours of administration. It would therefore not be appropriate for a patient to take a dosage of the faster-acting drugs such as ibuprofen less than 2 hours after the previous administration. The present disclosure addresses the technological problem of the generation of false reminders prompting a patient to break their adherence to the proper medication schedule (for example, by “reminding” the patient to take ibuprofen less than 2 hours after the previous dose). Thus, a medication reminder device according to the present disclosure may be configured to measure the temperature of the patient and to obtain a temperature-time curve. The temperature-time curve is compared with the blood concentration pharmacokinetics curve of the drug. The medication reminder device of the present disclosure is configured to generate a medication reminder only when the blood concentration of the drug falls below a critical threshold.

In other words, the medication reminder method of the present disclosure generates a reminder when a patient parameter is outside of a desired range and the drug concentration in the patient's blood serum falls below a threshold. A lower-than-threshold drug concentration in the blood serum signifies that that the level of the drug in the patient's body is no longer sufficient to maintain the patient parameter within the desired range. An advantage of linking the generation of a reminder to the concentration of the drug in the patient's blood serum is to reduce the risk of false reminders as a result of temporary fluctuations in the patient parameter (for example, during periods of exercise or other body movements).

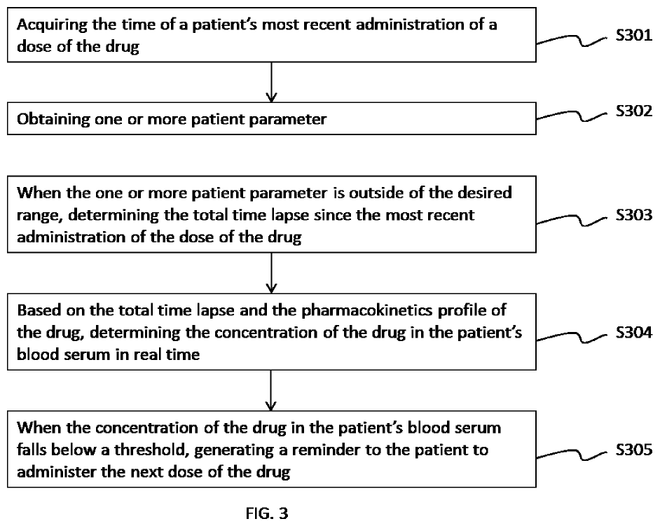

FIG. 3 is a flow diagram of a medication reminder method according to another embodiment of the present disclosure.

In step 301 (S301), the medication reminder device acquires the time of the most recent administration of a dose of the drug. After the patient is administered a dose of the drug, the patient can log the administration by pressing a designated button on the medication reminder device or by selecting a designated user-selectable item on a user interface of the medication reminder device. When the medication reminder device detects the input from the patient, the medication reminder device records and stores the time when the input is received as the time of the most recent administration of the drug dose.

Referring back to FIG. 1b, when the medication reminder device 101 is provided in a portable device, the medication reminder device 101 may be coupled to a wearable device 101a. After the patient is administered a dose of the drug, the patient can press a designated button on the wearable device 101a, or select a designated user-selectable item on the user interface of the wearable device 101a. When the wearable device 101a detects the input from the patient, the wearable device 101a may transmit the input to the medication reminder device 101. The medication reminder device 101 may in turn record and store the time when the input is received from the wearable device 101a as the time of the most recent administration of the drug dose.

Alternatively, referring back to FIG. 1c, when the medication reminder device 101 is provided in a portable device, the medication reminder device 101 may be coupled to a protective housing 101b. The protective housing 101b houses the medication reminder device 101. After the patient is administered a dose of the drug, the patient can press a designated button on the protective housing 101b. When the protective housing 101b detects the input from the patient, the protective housing 101b may transmit the input to the medication reminder device 101. The medication reminder device 101 may in turn record and store the time when the input is received from the protective housing 101b as the time of the most recent administration of the drug dose.

In some embodiments, the medication reminder device may designate the time of the most recent reminder as the time of the most recent administration of the drug dose.

In step 302 (S302), after a patient is administered a dose of a drug, the medication reminder device obtains one or more patient parameter. The patient parameter is used to characterize the medical condition being treated by the drug.

In some embodiments, after the medication reminder device has generated a reminder to the patient to administer the next dose of the drug, the medication reminder device may be configured to verify the proper administration of the proper dose. The verification may also occur after the medication reminder device has received the input from the patient signaling an administration of a dose of the drug. After the medication reminder device has verified the proper administration of the proper dose, the medication reminder device may be configured to monitor and obtain readings of the patient parameter, for example, periodically at regular intervals or in real time. The patient parameter being monitored is used to characterize the medical condition being treated by the drug.

As described above, in some embodiments, the medication reminder device 101 may be a portable device (for example, a mobile phone, a tablet, and the like) or a wearable device (for example, a smart bracelet, a smart watch, smart glasses, other wearable electronics that can be worn on the body as implants or accessories, and the like). When the medication reminder device 101 is provided in a wearable device, the wearable device comprises a sensor that is configured to monitor one or more patient parameter. When the medication reminder device 101 is provided in a portable device, the portable device comprising the medication reminder device 101 may in turn be coupled to a wearable device 101a or a protective housing 101b. The wearable device 101a and the protective housing 101b may comprise a sensor that is configured to monitor the patient parameter, for example, as shown in FIGS. 1b and 1c. The sensor of the wearable device 101a or the protective housing 101b may be used to acquire readings of the patient parameter, and to transmit the readings to the medication reminder device 101. For instance, when the patient is carrying the portable device comprising the medication reminder device 101, the sensor of the protective housing 101b may obtain readings of the patient parameter and transmit the readings to the medication reminder device 101.

In the embodiments shown in FIGS. 1b and 1c, the wearable device 101a or the protective housing 101b is not required to transmit real time readings of the patient parameter to the portable device comprising the medication reminder device 101. In some embodiments, the wearable device 101a or the protective housing 101b may transmit the readings at regular intervals, which may help conserve the power of the wearable device 101a or the protective housing 101b.

When the medical condition being treated can be characterized by a plurality of parameters, the medication reminder device may be configured to acquire readings of each of the plurality of parameters. In some embodiments, the medication reminder device may acquire readings of only the key parameters among the plurality of parameters.

The drugs that can be the target of the medication reminder method and system of the present disclosure are not particularly limited.

In some embodiments, the drug may be for controlling blood pressure. For example, the drug may be formulated to lower a patient's blood pressure, in which case the parameter to be monitored is the patient's blood pressure and the sensor for monitoring the patient parameter may be a blood pressure monitor.

In some embodiments, the drug may be for controlling blood sugar levels. For example, the drug may anti-diabetic medication formulated to lower blood glucose levels, in which case the parameter to be monitored is the patient's blood glucose level and the sensor for monitoring the patient parameter may be a non-invasive blood glucose meter. In some embodiments, the non-invasive blood glucose meter may comprise an infrared light emitting unit, an infrared light receiving unit, and a processor. The infrared light emitting unit may be configured to emit infrared light in the direction of a patient's hand or wrist. The infrared light receiving unit may be configured to receive infrared light from the infrared light emitting unit, which has penetrated the patient's body. Infrared light is known to be absorbed by glucose in blood, so that the intensity of the infrared light received by the infrared light receiving unit is expected to be lower than the intensity of the infrared light originally emitted by the infrared light emitting unit. The processor can calculate the difference between the intensity of the light emitted by the emitting unit and that of the light received by the receiving unit. Based on this difference, the patient's blood glucose can be determined. Since infrared light is absorbed by glucose in the patient's blood, there is a direct relationship between the level of the patient's blood glucose and the amount of infrared light absorption, and by calculating the difference in the intensities of the emitted and received infrared light, and comparing the calculated difference against a correlation between intensity difference and blood glucose level, which correlation may be predetermined, the non-invasive blood glucose meter can determine the level of blood glucose in the patient.

Returning to FIG. 3, in step 303 (S303), when the patient parameter is outside of a desired range, the medication reminder device determines the total time lapse since the previous administration of a dose of the drug.

After the medication reminder device has obtained readings of the patient parameter, the medical reminder device can determine whether the patient parameter is outside of a desired range. The range may correspond to range in a normal healthy individual, or to range selected by the patient themselves. For example, when the parameter being monitored is blood pressure, the range may be 90 mmHg to 140 mmHg. When the medication reminder device has acquired readings for a plurality of parameters that can be used to characterize the medical condition being treated, the medication reminder device may be configured to determine whether the acquired reading of each of the plurality of parameters is outside a corresponding range.

In some embodiments where only one parameter is being monitored, the medication reminder device may be configured to determine the total time lapse since the previous administration of a dose of the drug, when that parameter is outside the corresponding desired range. In some embodiments where a plurality of parameters are being monitored, the medication reminder device may be configured to determine the total time lapse since the previous administration of a dose of the drug, when each of the plurality of parameters being monitored is outside the corresponding desired range, or when one or more of the plurality of parameters being monitored is outside the corresponding range, and/or when the number of parameters that are outside their corresponding ranges exceeds a threshold value.

In S303, to determine the total time lapse since the previous administration of a dose of the drug, the medication reminder device may be configured to calculate the difference between the current time and the time when a dose of the drug was most recently administered.

In step 304 (S304), the medication reminder device determines the concentration of the drug in the patient's blood serum, in real time, based on the total time lapse since the previous administration of a dose of the drug and the pharmacokinetics profile of the drug.

The pharmacokinetics profile of a drug may be represented as a set of curves, a set of statistical data, or a mathematical formula. In practice, the pharmacokinetics profile of a drug is usually obtained from clinical trials.

In the present disclosure, the concentration of the drug in the patient's blood serum may be determined according to any one of the following three methodologies.

In the first methodology, the medication reminder device is configured to acquire the patient's medication information, which includes at least the identifying information of the drug. The medication reminder device is configured to then transmit a request to the server, requesting the pharmacokinetics profile of the drug. The request includes the identifying information of the drug. When the server receives the pharmacokinetics profile request from the medication reminder device, the server is configured to retrieve the pharmacokinetics profile corresponding to the identifying information contained in the request, and transmit the retrieved pharmacokinetics profile to the medication reminder device. The medication reminder device is configured to determine the concentration of the drug in the patient's blood based on the total time lapse since the previous administration of a dose of the drug and the pharmacokinetics profile of the drug.

In the second methodology, the medication reminder device is configured to acquire the patient's medication information, which includes at least the identifying information of the drug. The medication reminder device is configured to then transmit a request to the server, requesting determination of the concentration of the drug in the patient's blood. The request includes the identifying information of the drug, as well as the total time lapse since the previous administration of a dose of the drug. When the server receives the drug concentration determination request from the medication reminder device, the server is configured to retrieve the pharmacokinetics profile corresponding to the identifying information contained in the request. The server is configured to then determine the concentration of the drug in the patient's blood based on the total time lapse since the previous administration of a dose of the drug and the pharmacokinetics profile of the drug. The server is configured to transmit, and the medication reminder device is configured to receive, the drug concentration determination.

In the third methodology, the medication reminder device is configured to acquire the patient's medication information, which includes at least the identifying information of the drug. According to the identification information of the drug, the medication reminder device is configured to retrieve the corresponding pharmacokinetics profile of the drug from a preloaded database containing sets of identification information and corresponding pharmacokinetics profile for a plurality of drugs. The medication reminder device is configured to then determine the concentration of the drug in the patient's blood based on the total time lapse since the previous administration of a dose of the drug and the pharmacokinetics profile of the drug.

In some embodiments, the preloaded database in the medication reminder device is prepared by and transmitted from the server. For instance, when a doctor prescribes a drug for the patient, the doctor may transmit the prescription information from the doctor's terminal to the server. The prescription information may include the identifying information of the drug. When the server has received the prescription information, the server may retrieve the corresponding pharmacokinetics profile of the drug, and then transmit the drug's identifying information and pharmacokinetics profile to the medication reminder device specifically associated with the patient.

In some cases, patients of different ages and/or genders may metabolize the same drug differently, so that the same drug may have different pharmacokinetics profiles in different patients. In some embodiments of the present disclosure, the patient's medication information acquired by the medication reminder device may include, in addition to identifying information of the drug, physical information of the patient. The physical information may include at least one of age and gender of the patient. When the medication reminder device transmits a request to the server, requesting either the pharmacokinetics profile of the drug or determination of the blood concentration of the drug, the request may additionally include the patient's physical information. The server, or the medication reminder device itself, may retrieve the corresponding pharmacokinetics profile according to the drug's identifying information and the patient's physical information. The embodiments of the present disclosure thus make it possible to more accurately determine the blood concentration of the drug in a patient at any given moment, so as to further increase the accuracy of the medication reminders being generated.

In step 305 (S305), when the concentration of the drug in the patient's blood serum falls below a threshold, the medication reminder device is configured to generate a reminder to the patient to administer the next dose of the drug.

A lower-than-threshold drug concentration in the blood serum signifies that that the level of the drug in the patient's body is no longer sufficient to maintain the patient parameter within the desired range. An advantage of linking the generation of a reminder to the concentration of the drug in the patient's blood serum is to reduce the risk of false reminders as a result of temporary fluctuations in the patient parameter (for example, during periods of exercise or other body movements).

In some embodiments of the present disclosure, the medication reminder device is configured to generate a reminder in the form of a sound, a vibration, and/or a textual and/or graphical message containing the reminder. In some embodiments, the medication reminder device is configured to retrieve an image of the packaging and/or label of the drug, and to display the packaging and/or label in the textual and/or graphical message containing the reminder, so as to allow the patient to directly verify the proper drug to take and avoid administration of the wrong drugs.

Referring back to FIG. 1c, when the medication reminder device 101 is provided in a portable device, the medication reminder device 101 may be coupled to a protective housing 101b. In some embodiments, the protective housing 101b may comprise a holder for holding the drug. When the portable device has reminded the patient that it is time to take the drug, the patient can retrieve the drug from the holder in the protective housing 101b. In some embodiments, concurrently as the reminder to the patient to take the drug, the portable device may be configured to transmit a signal to the protective housing 101b to open the holder. Upon receipt of the signal, the protective housing 101b opens the holder, so as to remind the patient to take the drug inside the holder. In the modern age, a patient is more likely than not to carry a portable device, so that providing a holder in the protective housing 101b of the portable device would obviate the need for the patient to carry a separate pill box, which can in return eliminate the risk of the patient's non-adherence to the medication schedule as a result of having forgotten the pill box.

Embodiments of the medication reminder method and system described in the present disclosure determine whether a patient parameter is outside of a desired range, and then use this information to determine the total time lapse since a dose of the drug was previously administered. The concentration of the drug in the patient's blood serum is then determined based on the total time lapse. If the drug concentration in the patient's blood serum falls below a threshold, the medication reminder system reminds the patient to administer a dose of the drug. In other words, the medication reminder method and system of the present disclosure generate a reminder when the patient parameter is outside of the desired range and the drug concentration in the patient's blood serum falls below the threshold. A lower-than-threshold drug concentration in the blood serum signifies that that the level of the drug in the patient's body is no longer sufficient to maintain the patient parameter within the desired range. An advantage of linking the generation of a reminder to the concentration of the drug in the patient's blood serum is to reduce the risk of false reminders as a result of temporary fluctuations in the patient parameter (for example, during periods of exercise or other body movements)

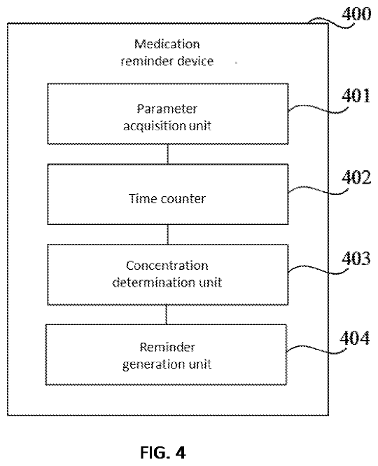

FIG. 4 is a block diagram of a medication reminder device according to an embodiment of the present disclosure. The medication reminder device 400 includes a parameter acquisition unit 401, a time counter 402, a concentration determination unit 403, and a reminder generation unit 404.

The parameter acquisition unit 401 is configured to, after a patient has been administered a dose of the drug, acquire readings of one or more patient parameter. The patient parameter is used to characterize the medical condition being treated by the drug. The parameter acquisition unit 401 is further configured to determine whether the acquired readings of the patient parameter is outside of the corresponding range.

The time counter 402 is configured to determine, when one or more of the acquired patient parameters is outside of a desired range, the total time lapse since the previous administration of a dose of the drug. This determination is made by quantifying the interval between the current time and the time when a dose of the drug was previously administered.

The concentration determination unit 403 is configured to determine the concentration of the drug in the patient's blood serum, based on the total time lapse since the previous administration of a dose of the drug and the pharmacokinetics profile of the drug.

In some embodiments, the concentration determination unit 403 is configured to acquire the patient's medication information, which includes at least the identifying information of the drug. The concentration determination unit 403 is configured to then transmit a request to the server, requesting the pharmacokinetics profile of the drug. The request includes the identifying information of the drug. The server receives the pharmacokinetics profile request from the concentration determination unit 403, retrieves the pharmacokinetics profile corresponding to the identifying information contained in the request, and transmits the retrieved pharmacokinetics profile to the concentration determination unit 403. The concentration determination unit 403 is configured to determine the concentration of the drug in the patient's blood based on the total time lapse since the previous administration of a dose of the drug and the pharmacokinetics profile of the drug.

In some embodiments, the concentration determination unit 403 is configured to acquire the patient's medication information, which includes at least the identifying information of the drug. The concentration determination unit 403 is configured to then transmit a request to the server, requesting determination of the concentration of the drug in the patient's blood. The request includes the identifying information of the drug, as well as the total time lapse since the previous administration of a dose of the drug. The server receives the pharmacokinetics profile request from the medication reminder device, retrieves the pharmacokinetics profile corresponding to the identifying information contained in the request, calculates the concentration of the drug in the patient's blood based on the total time lapse since the previous administration of a dose of the drug and the pharmacokinetics profile of the drug, and then transmits drug concentration determination to the concentration determination unit 403.

In some embodiments, the concentration determination unit 403 is configured to acquire the patient's medication information, which includes at least the identifying information of the drug. According to the identification information of the drug, the concentration determination unit 403 is configured to retrieve the corresponding pharmacokinetics profile of the drug from a preloaded database containing sets of identification information and corresponding pharmacokinetics profile for a plurality of drugs. The concentration determination unit 403 is configured to then determine the concentration of the drug in the patient's blood based on the total time lapse since the previous administration of a dose of the drug and the pharmacokinetics profile of the drug.

In some embodiments, the reminder generation unit 404 is configured to retrieve an image of the packaging and/or label of the drug, and to display the packaging and/or label, for example, in the reminder.

The drugs that can be the target of the medication reminder device of the present disclosure are not particularly limited. In some embodiments, the drug may be for controlling blood pressure. For example, the drug may be formulated to lower a patient's blood pressure, in which case the parameter acquisition unit 401 may include a blood pressure monitor, which is configured to monitor and acquire readings of the patient's blood pressure.

In some embodiments, the drug may be for controlling blood sugar levels. For example, the drug may anti-diabetic medication formulated to lower blood glucose levels, in which case the parameter acquisition unit 401 may include a non-invasive blood glucose meter, which is configured to monitor and acquire readings of the patient's blood glucose level. In some embodiments, the non-invasive blood glucose meter may comprise an infrared light emitting unit, an infrared light receiving unit, and a processor. The infrared light emitting unit may be configured to emit infrared light in the direction of a patient's hand or wrist. The infrared light receiving unit may be configured to receive infrared light from the infrared light emitting unit, which has penetrated the patient's body. Infrared light is known to be absorbed by glucose in blood, so that the intensity of the infrared light received by the infrared light receiving unit is expected to be lower than the intensity of the infrared light originally emitted by the infrared light emitting unit. The processor can calculate the difference between the intensity of the light emitted by the emitting unit and that of the light received by the receiving unit. Based on this difference, the patient's blood glucose can be determined.

In some embodiments, the medication reminder device 404 may be provided in a portable device (for example, a mobile phone, a tablet, and the like), which may be coupled to a wearable device (for example, as illustrated in FIG. 1b). The wearable device may include a sensor that is configured to monitor a patient parameter. In those cases, the parameter acquisition unit 401 of the medication reminder device 404 may be configured to monitor and acquire readings of the patient parameter via the wearable device.

In some embodiments where the medication reminder device 404 is provided in a portable device, the portable device containing the medication reminder device 404 may be coupled to a protective housing (for example, as illustrated in FIG. 1c). The protective housing houses the portable device comprising the medication reminder device 101, and may comprise a sensor that is configured to monitor a patient parameter. In those cases, when the patient is carrying the portable device comprising the medication reminder device 404, the parameter acquisition unit 401 may be configured to monitor and acquire readings of the patient parameter via the sensor of the protective housing. In some embodiments, the protective housing may comprise a holder for holding the drug.

In some embodiments, the medication reminder device 400 may be provided in a wearable device (for example, a smart bracelet, a smart watch, smart glasses, other wearable electronics that can be worn on the body as implants or accessories, and the like). When the medication reminder device 400 is provided in a wearable device, the wearable device may comprise a sensor that is configured to monitor a patient parameter.

The medication reminder device 400 may be any configuration of hardware and/or software known to a person of ordinary skill in the art to be capable of executing the medication reminder method described in the present disclosure. For example, the medication reminder device 400 may be configured as a microprocessor, a digital signal processor, an application specific integrated circuit, any suitable programmable logic device, discrete hardware components, or any combination thereof, which may be designed to perform the functions described herein. That is, there are no particular limitations on the implementation forms of the medication reminder device 400.

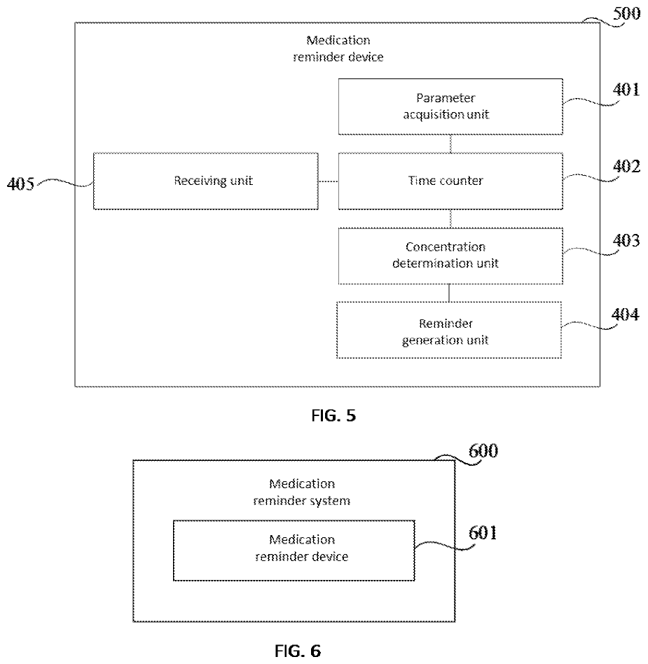

FIG. 5 is a block diagram of a medication reminder device according to another embodiment of the present disclosure. The medication reminder device 500 includes the same components as the medication reminder device 400 illustrated in FIG. 4, but additionally, the medication reminder device 500 includes a receiving unit 405.

The receiving unit 405 is configured to receive an input corresponding to the patient's most recent administration of a dose of the drug, and record and store the time of receipt of the input. The time counter 402 is configured to determine the total time lapse since the previous administration of a dose of the drug, by quantifying the time interval between the current time and the recorded time of receipt of the input at the receiving unit 405.

Embodiments of the medication reminder device described in the present disclosure determine whether a patient parameter is outside of a desired range, and then use this information to determine the total time lapse since a dose of the drug was previously administered. The concentration of the drug in the patient's blood serum is then determined based on the total time lapse. If the drug concentration in the patient's blood serum falls below a threshold, the medication reminder system reminds the patient to administer a dose of the drug. In other words, the medication reminder device of the present disclosure generates a reminder when the patient parameter is outside of the desired range and the drug concentration in the patient's blood serum falls below the threshold. A lower-than-threshold drug concentration in the blood serum signifies that that the level of the drug in the patient's body is no longer sufficient to maintain the patient parameter within the desired range. An advantage of linking the generation of a reminder to the concentration of the drug in the patient's blood serum is to reduce the risk of false reminders as a result of temporary fluctuations in the patient parameter (for example, during periods of exercise or other body movements).

FIG. 6 is a block diagram of a medication reminder system 600 according to an embodiment of the present disclosure. The medication reminder system 600 includes a medication reminder device 601. The medication reminder device 601 is configured as described in the present disclosure, for example, as illustrated in FIG. 4 or FIG. 5. The medication reminder 601 is configured to perform a medication reminder method in the manner described in the present disclosure, for example, as illustrated in FIG. 2 or FIG. 3.

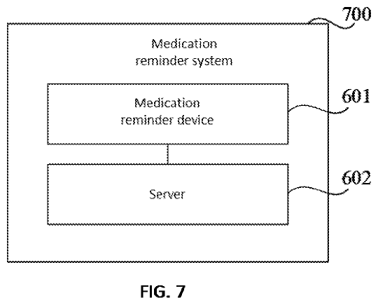

FIG. 7 is a block diagram of a medication reminder system 700 according to another embodiment of the present disclosure. The medication reminder system 700 includes a medication reminder device 601 and a server 602. The server 602 is configured as described in the present disclosure, for example, as illustrated in FIG. 3.

In some embodiments, the server 602 is configured to receive, from the medication reminder device 601, a pharmacokinetics profile request for a particular drug. The pharmacokinetics profile request includes the identifying information of the drug. The server 602 is configured to then retrieve the pharmacokinetics profile corresponding to the identifying information contained in the request, and transmit the retrieved pharmacokinetics profile to the medication reminder device 601. The medication reminder device 601 is configured to determine the concentration of the drug in the patient's blood based on the total time lapse since the previous administration of a dose of the drug and the pharmacokinetics profile of the drug.

In some embodiments, the server 602 is configured to receive, from the medication reminder device 601, a request to determine the concentration of a particular drug in the patient's blood serum. The request includes the identifying information of the drug, as well as the total time lapse since the previous administration of a dose of the drug. The server 602 is configured to then retrieve the pharmacokinetics profile corresponding to the identifying information contained in the request, determine the concentration of the drug in the patient's blood based on the total time lapse since the previous administration of a dose of the drug and the pharmacokinetics profile of the drug, and then transmit drug concentration determination to the medication reminder device 601.

In some embodiments, the medication reminder device 601 may be provided in a portable device (for example, a mobile phone, a tablet, and the like) or a wearable device (for example, a smart bracelet, a smart watch, smart glasses, other wearable electronics that can be worn on the body as implants or accessories, and the like). When the medication reminder device 601 is provided in a portable device, the medication reminder system 600 may include a wearable device coupled to the medication reminder device 601, or a protective housing coupled to the medication reminder device 601. The wearable device and the protective housing may comprise a sensor that is configured to monitor a patient parameter. In some embodiments, the protective housing may additionally comprise a holder for holding the drug.

Embodiments of the medication reminder system described in the present disclosure determine whether a patient parameter is outside of a desired range, and then use this information to determine the total time lapse since a dose of the drug was previously administered. The concentration of the drug in the patient's blood serum is then determined based on the total time lapse. If the drug concentration in the patient's blood serum falls below a threshold, the medication reminder system reminds the patient to administer a dose of the drug. In other words, the medication reminder system of the present disclosure generates a reminder when the patient parameter is outside of the desired range and the drug concentration in the patient's blood serum falls below the threshold. A lower-than-threshold drug concentration in the blood serum signifies that that the level of the drug in the patient's body is no longer sufficient to maintain the patient parameter within the desired range. An advantage of linking the generation of a reminder to the concentration of the drug in the patient's blood serum is to reduce the risk of false reminders as a result of temporary fluctuations in the patient parameter (for example, during periods of exercise or other body movements).

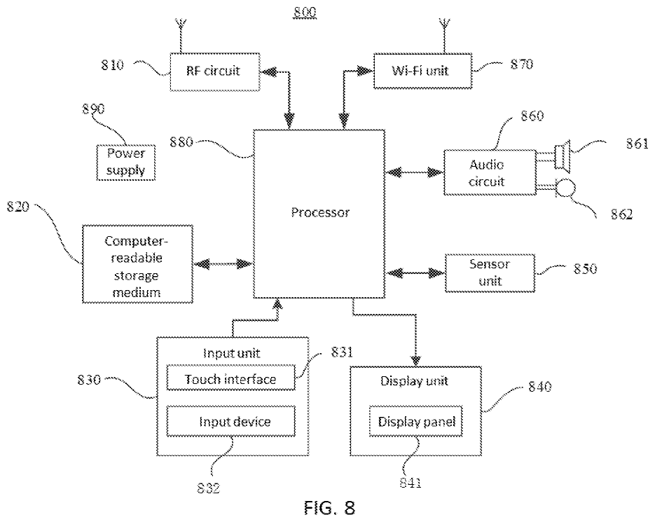

FIG. 8 is a schematic diagram of a terminal device according to an embodiment of the present disclosure. A medication reminder device configured according to the present disclosure may be provided in the terminal device. As shown in FIG. 8, the terminal device 800 may include at least a radio frequency (RF) circuit 810, at least one non-transitory computer-readable storage medium 820 such as a memory device or random access memory, an input unit 830, a display unit 840, a sensor unit 850, audio circuit 860, wireless fidelity (Wi-Fi) unit 870, at least one core processor unit (CPU) 880, and a power supply 890. However, FIG. 8 provides only a non-limiting example of a terminal device according to the present disclosure, and the components of the terminal device are not limited to those shown in FIG. 8. A terminal device according to the present disclosure may be configured and arranged in any suitable manner known to a person of ordinary skill in the art.

In some embodiments, the RF circuit 810 may be configured to receive and send signals transmitting a message or a call. For example, the RF circuit 810 may be configured to transmit downlink communication signals received from a base station to the processor(s) 880 for processing, and to transmit uplink communication signals to the base station. Generally, the RF circuit 810 includes, but is not limited to, an antenna, at least one amplifier, a tuner, at least one oscillator, a subscriber identity module (SIM) card, a transceiver, a coupler, a low noise amplifier (LNA), and a diplexer. However, the configuration and arrangement of the RF circuit according to the present disclosure are not particularly limited, and may be devised in any suitable manner known to a person of ordinary skill in the art. The RF circuit 810 may also connect to the internet or other devices via a wireless connection. The wireless connection is not particularly limited, and may adopt any standard or protocol known to a person of ordinary skill in the art, including, but not limited to, Global System for Mobile Communications (GSM), General Packet Radio Service (GPRS), Code Division Multiple Access (CDMA), Wideband Code Division Multiple Access (WCDMA), Long Term Evolution (LTE), electronic mail, Short Messaging Service (SMS), and the like.

The non-transitory computer-readable storage medium 820 may be configured to store software programs and modules. The processor(s) 880 executes a variety of functional applications and data processing by running the software programs and modules stored in the computer-readable storage medium 820. The computer-readable storage medium 820 may include a program storage area and a data storage area. The program storage area may be configured to store a variety of software programs, including an operating system, at least one software application required to perform the functions of the terminal device (for example, a sound playback function and an image playback function), and the like. The storage data area may be configured to store a variety of data, including data created during the use of the terminal device 800 (for example, audio data, contact list, and the like). The computer-readable storage medium 820 may also include a high-speed random access memory, and also a non-volatile memory. For example, the readable storage medium 820 may include at least one magnetic disk storage device, flash memory device, or other non-volatile solid-state storage devices. The computer-readable storage medium 820 may further include a memory controller configured to provide access to the computer-readable storage medium 820 by the processor 880 and the input unit 830.

The input unit 830 may be configured to receive input of letter and/or alphanumeric data, and to generate signal input for a keyboard, a mouse, a joystick, and/or an optical or trackball that relates to user settings and/or function control. The input unit 830 may include a touch-sensitive user interface 831 and other input devices 832. The touch-sensitive user interface 831, also referred to as a touchscreen or touchpad, may be configured to collect touch operations on or near the interface by the user (for example, the user may use finger, stylus, and any other suitable instrument or accessory to operate on the touch-sensitive user interface 831), and to drive the corresponding connecting devices according to a preset applications. The touch-sensitive user interface 831 may include a touch detection device and a touch controller. The touch detection device is configured to detect the position of the touch input from the user, detect the signal generated by the touch input, and send the signal to the touch controller. The touch controller is configured to receive the signal from the touch detection device, and to convert the signal into position coordinates, which are then sent to the processor(s) 880. The touch controller is also configured to receive and execute commands received from the processor(s) 880. In addition, the touch-sensitive user interface 831 can be implemented in any suitable form known to a person of ordinary skill in the art, including as a resistive, capacitive, infrared, or surface acoustic wave touchscreen. In addition to the touch-sensitive user interface 831, the input unit 830 may include any additional, suitable input devices 832 known to a person of ordinary skill in the art. For example, the additional input devices 832 may include, but are not limited to, at least one physical keyboard, functional keys (for example, volume control keys, switch keys, and the like), trackballs, mouse, joysticks, and the like.

The display unit 840 may be configured to display information input by the user, information to be provided to the user, and/or to serve as graphical user interfaces of the terminal device 800. The graphical user interfaces may be formed of graphics, text, icons, videos, and any combination thereof. The display unit 840 may include a display panel 841, which may be a liquid crystal display (LCD), an organic light-emitting diode (OLED) display, and the like. In some embodiments, the touch-sensitive user interface 831 may cover the display panel 841. When the touch-sensitive user interface 831 detects a touch input on or near the interface 831, the interface 831 transmits the input to the processor(s) 880 to decode the touch input, after which the processor(s) 880 generate a visual output on the display panel 841 based on the nature of the touch input. As shown in FIG. 8, the touch-sensitive user interface 831 and the display panel 841 are separate components, but in some embodiments, the touch-sensitive user interface 831 and the display panel 841 may be an integrated unit, so as to enable dual input and output functions.

The terminal device 800 may also include at least one sensor 850, such as a light sensor, a motion sensor, and the like. The light sensor may include an ambient light sensor and a proximity sensor. The ambient light sensor may be configured to adjust the brightness of the display panel 841 according to the intensity of the ambient light. The proximity sensor may be configured to turn off the display panel 841 and/or the backlight when the terminal device 800 is moved to the ear of the user. The motion sensor may be a gravity sensor. The gravity sensor may be configured to detect gravity acceleration in multiple directions (usually along the x, y, and z axes), and when stationary, may be configured to detect the magnitude and direction of the gravity acceleration. The gravity sensor may be used to identify the tilt of the portable device during different applications (for example, horizontal and vertical screen orientation, related games, magnetometer attitude calibration), to enable functions involving vibration-based recognition (such as pedometer, tapping), and the like. The terminal device 800 may further include a gyroscope, a barometer, a hygrometer, a thermometer, an infrared sensor, and any other suitable components known to a person of ordinary skill in the art.

The audio circuit 860, the speaker 861, and the microphone 861 provide an auditory interface between the user and the terminal device 800. The audio circuit 860 may be configured to receive and convert audio data into electrical signals, and to transmit the electrical signals to the speaker 861 to be converted into audio signals by the speaker 861. The microphone 862 converts the acquired sound signals into electrical signals, which are then transmitted to the audio circuit 860 and converted into audio data. The audio data are transmitted to the processor(s) 880 processor(s) 880 for processing, after which the audio data are either transmitted along the RF circuit 810 to another terminal, or are output to the computer-readable storage medium 820 for further processing. The audio circuit 860 may also include an outlet for coupling a peripheral headset and the terminal device 800.

Wi-Fi relates to short-range wireless transmission technology, and through the Wi-Fi unit 870, the terminal device 800 may be configured to provide a user with wireless broadband internet access, so as to enable functions such as emails, web browsing, multimedia streaming, and the like. FIG. 8 shows an embodiment of a medication reminder system of the present disclosure that includes the Wi-Fi unit 870, but it is understood that the Wi-Fi unit 870 may be omitted from the medication reminder system without departing from the spirit and scope of the present disclosure.

The processor 880 is the control center of the terminal device 800, and connects the various components of the terminal device 800 via interfaces and/or circuits. The processor 880 is configured to perform the myriad functions and/or data processing of the terminal device 800, as well as monitoring the overall operations of the terminal device 800, by executing software programs and/or applications stored in the computer-readable storage medium 820 and/or by retrieving data stored in the computer-readable storage medium 820.

In some embodiments, the processor 880 may include one or more core processors. In a preferred embodiment, the processor 880 includes an integrated application processor and baseband processor. The application processor delivers all system capabilities needed to support the terminal device 800, including running the operating system, user interface, software applications, and the like. The baseband processor functions mainly to manage wireless communications. It is understood that the baseband processor may be omitted from the processor 880 in a medication reminder system of the present disclosure without departing from the spirit and scope of the present disclosure.

The terminal device 800 may also include a power supply 890 (for example, a battery) for powering each component of the terminal device 800. In some embodiments, the power supply 890 may be logically connected to the processor 880 through a power management system, and via the power management system, manage such functions as charging, discharging, and power consumption management. The power supply 890 may also include one or more DC or AC power sources, a recharging system, a power failure detection circuit, a power converter or an inverter, a power status indicator, and the like.

The terminal device 800 may also include a camera, a Bluetooth module, and other suitable components or accessories known to a person of ordinary skill in the art. In some embodiments, the display unit of the terminal device is a touch screen display, and the terminal device includes a memory, at least one software program stored in the memory and processed by the processor.

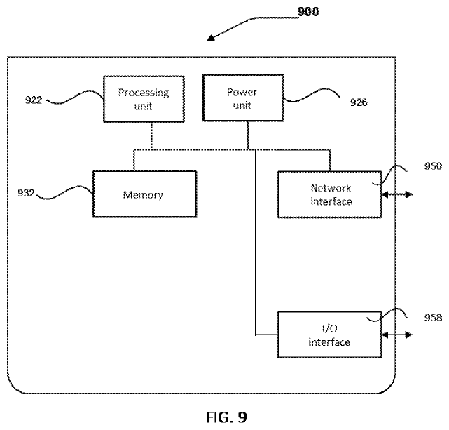

FIG. 9 shows a schematic diagram of a server according to an embodiment of the present disclosure. The server is configured as described in the present disclosure, including performing the medication reminder method described in the present disclosure, for example, as illustrated in FIG. 3. As shown in FIG. 9, the server may include a processing unit 922. The processing unit 922 may include at least one processor and a storage medium represented by the memory 932, which storage medium is configured to store commands (for example, software applications) to be executed by the processing unit 922. The software applications stored in the memory 932 may include at least module each corresponding to a set of commands. The processing unit 922 is configured to execute those commands.

The server 900 may also include a power unit 926 that is configured to undertake power management of the server 900; a wired or wireless network interface 950 that is configured to connect the server 900 to a network; and an input/output (I/O) interface 958. The server 900 may run on an operating system stored in the memory 932. The operating system may be Windows Server™, Mac OS X™, Unix™, Linux™, FreeBSD™, or any suitable operating system known to a person of ordinary skill in the art.

In another aspect of the present disclosure, a computer-readable storage medium is provided, which is configured to store software programs, including software program(s) for executing a medication reminder method according to the present disclosure. The computer-readable storage medium may be non-transitory. Executing software program(s) stored in the computer-readable storage medium may execute a medication reminder method according to the present disclosure. In some embodiments, the computer-readable storage medium is configured to store software program(s) that obtains, after a patient is administered a dose of a drug, the patient parameter, wherein the patient parameter is used to characterize the medical condition being treated by the drug; when the patient parameter is outside of a desired range, determines the total time lapse since the previous administration of a dose of the drug; based on the total time lapse, determines the concentration of the drug in the patient's blood serum in real time; and when the concentration of the drug in the patient's blood serum falls below a threshold, generates a reminder to the patient to administer the next dose of the drug.

A person of ordinary skill in the art readily understands that all or part of the steps of the medication reminder method of the present disclosure may be implemented by hardware, or by programming the applicable hardware with the proper software programs. The software programs may be stored in a computer-readable storage medium, which may be a read-only memory, a magnetic disk, or an optical disk.

It should be appreciated that changes could be made to the embodiments described above without departing from the inventive concepts thereof. It should be understood, therefore, that this invention is not limited to the particular embodiments disclosed, but it is intended to cover modifications within the spirit and scope of the present invention as defined by the appended claims.

What is claimed is:

1. A method for providing a reminder to a patient of when a second dose of a medication should be administered, the patient having been administered a first dose of the medication, the method comprising:
obtaining a measurement of at least one patient parameter;
determining whether the measurement of the at least one patient parameter is outside of a range;
when the measurement of the at least one patient parameter is outside of the range, determining a total time lapse since the administration of the first dose of the medication;
determining a concentration of the medication in blood of the patient based at least on the total time lapse; and
when the concentration of the medication in the blood of the patient is below a threshold, generating a reminder to the patient to administer the second dose of the medication,
wherein the at least one patient parameter characterizes a medical condition to be treated by the medication.

2. The method according to claim 1, further comprising retrieving a pharmacokinetics profile of the medication,
wherein the concentration of the medication in the blood of the patient is determined based on the total time lapse and the pharmacokinetics profile of the medication.

3. The method according to claim 2, further comprising obtaining a profile of the patient,
wherein the profile comprises information relating to at least one of an age and a gender of the patient, and
wherein the retrieved pharmacokinetics profile of the medication is specific to at least one of the age and the gender of the patient.

4. The method according to claim 1, further comprising receiving, at a time of the administration of the first dose of the medication, an indication indicating the time of the administration.

5. The method according to claim 4, further comprising determining the total time lapse based on the time of the administration of the first dose of the medication.

6. The method according to claim 1, further comprising obtaining an image of a packaging of the medication,
wherein the generated reminder comprises the image of the packaging of the medication.

7. A device for providing a reminder to a patient of when a second dose of a medication should be administered, the patient having been administered a first dose of the medication, the device comprising:
a parameter acquisition unit configured to obtain a measurement of at least one patient parameter, and determine whether the measurement of the at least one patient parameter is outside of a range;
a time counter configured to, when the measurement of the at least one patient parameter is outside of the range, determine a total time lapse since the administration of the first dose of the medication;
a concentration determination unit configured to determine a concentration of the medication in blood of the patient based at least on the calculated total time lapse; and
a reminder generation unit configured to, when the concentration of the medication in the blood of the patient is below a threshold, generate a reminder to the patient to administer the second dose of the medication,
wherein the at least one patient parameter characterizes a medical condition to be treated by the medication.

8. The device according to claim 7, wherein the concentration determination unit is further configured to acquire a pharmacokinetics profile of the medication, so as to determine the concentration of the medication in the blood of the patient based on the total time lapse and the pharmacokinetics profile of the medication.

9. The device according to claim 7, wherein the reminder generation unit is further configured to obtain an image of a packaging of the medication, and configured to generate a reminder that comprises the image of the packaging of the medication.

10. The device according to claim 7, further comprising a receiving unit configured to receive, at a time of the administration of the first dose of the medication, an indication indicating the time of the administration,
wherein the time counter is configured to determine the total time lapse based on the time of the administration of the first dose of the medication.

11. A system for providing a reminder to a patient of when a second dose of a medication should be administered, the patient having been administered a first dose of the medication, the system comprising:
the medication reminder device according to claim 7, and
a server coupled to the medication reminder device.

12. The system according to claim 11,
wherein the concentration determination unit is further configured to acquire medication information of the patient, the medication information including identifying information of the medication,
wherein the concentration determination unit is further configured to transmit to the server a request for a pharmacokinetics profile of the medication, the request including the identifying information of the medication,
wherein the server is configured to retrieve the pharmacokinetics profile of the medication based on the identifying information of the medication, and to transmit the pharmacokinetics profile of the medication to the concentration determination unit for determining the concentration of the medication in the blood of the patient.

13. The system according to claim 11,
wherein the concentration determination unit is further configured to acquire medication information of the patient, the medication information including identifying information of the medication,
wherein the concentration determination unit is further configured to transmit to the server a request for a determination of the concentration of the medication in the blood of the patient, the request including the identifying information of the medication and the total time lapse,
wherein the server is configured to:
retrieve a pharmacokinetics profile of the medication based on the identifying information of the medication,
determine the concentration of the drug in the blood of the patient based on the total time lapse and the pharmacokinetics profile of the medication, and
transmit the drug concentration determination to the medication reminder device.

14. The system according to claim 11,
wherein the concentration determination unit is further configured to:
acquire medication information of the patient, the medication information including identifying information of the medication,
based on the identifying information of the medication, retrieve a pharmacokinetics profile of the medication, and
determine the concentration of the drug in the blood of the patient based on the total time lapse and the pharmacokinetics profile of the medication.

15. The system according to claim 11,
wherein the medication reminder device is provided in a portable device which is coupled to a wearable device, and
wherein the wearable device comprises a sensor configured to measure the at least one patient parameter.

16. The system according to claim 11, wherein the medication reminder device is provided in a portable device which is coupled to a protective housing, the protective housing being configured to house the portable device, and
wherein the protective housing comprises a sensor configured to measure the at least one patient parameter.

17. A non-transitory computer-readable storage medium storing a program that, when executed by a computer, performs the method according to claim 1.
